(19) United States
(12) Patent Application Publication
(10) Pub. No.: US 2016/0367529 A1
(43) Pub. Date: Dec. 22, 2016

(54) DEBILITY PREVENTATIVE

(71) Applicant: AJINOMOTO CO., INC., Tokyo (JP)

(72) Inventors: Mika HAMADA, Kawasaki-shi (JP); Tomoyuki MINE, Kawasaki-shi (JP); Naoki HAYASHI, Kawasaki-shi (JP); Ryo UCHIDA, Kawasaki-shi (JP)

(73) Assignee: AJINOMOTO CO., INC., Tokyo (JP)

(21) Appl. No.: 15/255,906

(22) Filed: Sep. 2, 2016

Related U.S. Application Data
(63) Continuation of application No. PCT/JP2015/056725, filed on Mar. 6, 2015.

(30) Foreign Application Priority Data
Mar. 7, 2014 (JP) 2014-045247

(51) Int. Cl. A61K 31/405; A61K 9/00; A21D 2/24; A23L 33/00; A23K 20/147; A23L 2/39; A23L 2/52; A23L 33/175; A61K 31/198; A23G 1/42

(52) U.S. Cl. CPC A61K 31/405; A61K 31/198; A61K 9/0056; A61K 9/0095; A21D 2/245; A23G 1/42; A23K 20/147; A23L 2/39; A23L 2/52; A23L 33/175; A23L 33/40; A23V 2002/00

(57) ABSTRACT

Administering an agent containing isoleucine and threonine as active ingredients is effective for the prophylaxis or improvement of frailty in an elderly person and is safe even by ingestion for a long term. The agent for the prophylaxis or improvement of frailty may further contain tryptophan and/or methionine as an active ingredient.

### CROSS REFERENCES TO RELATED APPLICATIONS

This application is a continuation of International Patent Application No. PCT/JP15/056725, filed on Mar. 6, 2015, and claims priority to Japanese Patent Application No. 2014-045247, filed on Mar. 7, 2014, both of which are incorporated herein by reference in their entireties.

### BACKGROUND OF THE INVENTION

### Field of the Invention

The present invention relates to agents for the prophylaxis or improvement of frailty in an elderly person.

### Discussion of the Background

Rapid aging is proceeding, and an increase of health expectancy not only improves the quality of life (QOL) of elderly person, but is also desired for insurance of sustainable social life. To increase health expectancy, it is effective to prevent elderly person syndrome which is said to represent various physical and mental symptoms not necessarily considered illness, from among the symptoms that appear along with aging.

Particularly, “frailty” which is one of the elderly person syndromes is a condition before developing health problems (life dysfunction, etc.), which is generated by a decrease in the spare capacity of various organs that play an important role in life activity and the ability to adapt to changes in not only the internal environment of the body but also external environment. Since prophylaxis of frailty leads to prevention or delaying of transfer to a condition in need of nursing care and the like, increases health expectancy, also improves quality of life, and decreases elderly person requiring nursing care, the prophylaxis thereof has been desired.

On the other hand, a method using muscular exercise for the prophylaxis or improvement of frailty has been reported (see Yamada M, et al., Effect of resistance training on physical performance and fear of falling in elderly with different levels of physical well-being, Age Ageing, 40 (5): 637-641 (2011) which is incorporated herein by reference in its entirety). In muscular exercise (3 times per week) performed for person in need of nursing care (75.8±4.5 years old, n=324) as a target, the muscle mass did not increase in 3 months, and increased by 2.7% 9 months later and 5% one year later. Therefrom it is considered that exercise for a considerably long term is necessary for the recovery of muscle strength in elderly person with a small muscle mass.

Also, in a study examining the intervention effects in nutrition group, exercise (3 times per week) group, nutrition+exercise group of elderly person in residential care, who were 70 years or older, as the target for 10 weeks, the nutrition+exercise group showed the highest improvement in the muscle strength and motor function. However, it has been reported that nutrition reinforcement increases ingestion calorie and body weight, but does not improve muscle strength (see Fiatarone M A, et al., Exercise training and nutritional supplementation for physical frailty in very elderly person, N Engl J Med., 330 (25): 1769-1775 (1994) which is incorporated herein by reference in its entirety).

Furthermore, it is described that essential amino acid preparations accelerate recovery from physical fatigue and mental fatigue (see JP-A-9-52828, which is incorporated herein by reference in its entirety), and a diet composition containing essential amino acid and not containing protein defends animals and human from aging and the like (see National Publication of International Patent Application No. 2011-523626, which is incorporated herein by reference in its entirety). It is known that an oral amino acid-containing composition containing L-leucine can prevent or improve loss of skeletal muscle mass in elderly person (see National Publication of International Patent Application No. 2008-534599, which is incorporated herein by reference in its entirety). However, these do not improve muscle strength or motor function.

Under such situation, the development of an active ingredient which can improve muscle strength and motor function and prevent frailty without intervention of exercise and the like in elderly person with a small muscle mass, and which is safe even when taken for a long term has been desired.

### SUMMARY OF THE INVENTION

Accordingly, it is one object of the present invention to provide novel agents which improve muscle strength, muscle mass, and incentive of elderly person.

It is another object of the present invention to provide novel agents for the prophylaxis or improvement of frailty, which can be ingested safely for a long term.

It is another object of the present invention to provide novel methods for improving muscle strength, muscle mass, and incentive of elderly person.

It is another object of the present invention to provide novel methods for the prophylaxis or improvement of frailty, which can be ingested safely for a long term.

These and other objects, which will become apparent during the following detailed description, have been achieved by the inventors' discovery that a composition containing isoleucine and threonine improves various physical and mental symptoms and can prevent or improve frailty, and that a composition further containing tryptophan and/or methionine can prevent or improve frailty more.

Accordingly, the present invention provides the following:

(1) An agent for the prophylaxis or improvement of frailty, comprising isoleucine and threonine as active ingredients.

(2) The agent of (1), further comprising at least one kind of amino acid selected from tryptophan and methionine as an active ingredient.

(3) The agent of (1) or (2), wherein a weight ratio of isoleucine and threonine is 1:0.2 to 10.

(4) The agent of (2) or (3), wherein a weight ratio of tryptophan, threonine, methionine and isoleucine is 1:0.5 to 12:0.2 to 10:0.5 to 12.

(5) The agent of any one of (1)-(4), which is in a unit package form per serving comprising not less than 0.04 g, preferably not less than 0.06 g, of an amino acid as an active ingredient in an ingestion amount per one serving.

(6) The agent of any one of (1)-(5), wherein the frailty is at least one kind of symptom selected from the group consisting of loss of muscle mass, decline in grip strength, feeling of fatigue, decrease in walking speed, decrease in the amount of physical activity, loss of motivation, depression state, delirium, dementia, sleep disorder, anxiety disorder and social withdrawal, in elderly person.

(7) The agent of any one of (2)-(6), which is substantially free of an amino acid other than tryptophan, threonine, methionine, and isoleucine.

(8) A food or drink for the prophylaxis or improvement of frailty, which is in a unit package form per serving comprising not less than 0.04 g of isoleucine and threonine in total as an active ingredient at a weight ratio of isoleucine and threonine of 1:0.2 to 10.

(9) A food or drink for the prophylaxis or improvement of frailty, which is in a unit package form per serving comprising not less than 0.06 g of 4 kinds of amino acids of tryptophan, threonine, methionine and isoleucine in total as an active ingredient at a weight ratio of tryptophan, threonine, methionine and isoleucine of 1:0.5 to 12:0.2 to 10:0.5 to 12.

(10) A kit, comprising a measuring container and a food or drink for the prophylaxis or improvement of frailty, comprising isoleucine and threonine as active ingredients, wherein the measuring container is for measuring not less than 0.04 g in total of the aforementioned amino acid.

(11) A kit, comprising a measuring container and a food or drink for the prophylaxis or improvement of frailty, comprising 4 kinds of amino acids of tryptophan, threonine, methionine and isoleucine as active ingredients, wherein the measuring container is for measuring not less than 0.06 g in total of the above-mentioned 4 kinds of amino acids.

According to the present invention, muscle strength and muscle mass can be increased in elderly person without intervention of exercise, frailty can be prevented, and the QOL of elderly person can be enhanced by merely ingesting 2 to 4 kinds of amino acids of isoleucine and threonine, and further, tryptophan and/or methionine.

According to the present invention, incentives for activity are promoted, the amount of motor activity is increased, and muscle strength and muscle mass can be increased in elderly person by merely ingesting the aforementioned 2 to 4 kinds of particular amino acids.

According to the present invention, moreover, health problems of elderly person can be delayed, health expectancy can be extended, and the medical expenses and care costs can be suppressed.

Since amino acid is an active ingredient, frailty can be prevented by safely administering for a long term.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows a feed intake schedule for rat.

FIG. 2 shows an influence of tryptophan (Trp), threonine (Thr), methionine (Met) and isoleucine (Ile) (hereinafter to be also referred to as 4AA) on feed intake in low protein model rats.

FIG. 3 shows an influence of 4AA on spontaneous activity of low protein model rat.

FIG. 4 shows an influence of the addition of 4AA, or 4AA excluding Trp, Thr or Met on the body weight of low protein model rat.

FIG. 5 shows an influence of 4AA, Ile and Thr, Ile or Thr on the body weight of low protein model rat.

FIG. 6 shows an influence of the addition of 4AA; essential and non-essential amino acids excluding 4AA (ALL-4AA); essential amino acid excluding 4AA (EAA-4AA), non-essential amino acid (NEAA); glycine (Gly) on the body weight of low protein model rat.

FIG. 7 shows an influence of the addition of 4AA; essential and non-essential amino acids excluding 4AA (ALL-4AA); essential amino acid excluding 4AA (EAA-4AA), non-essential amino acid (NEAA); glycine (Gly) on the weight of viscera-isolated carcass of low protein model rat.

### DETAILED DESCRIPTION OF THE PREFERRED EMBODIMENTS

The present invention relates to an agent for the prophylaxis or improvement of frailty, comprising, as active ingredients, at least isoleucine and threonine, and further, tryptophan or methionine, and an agent for the prophylaxis or improvement of frailty, comprising 4 kinds of amino acids of (1) tryptophan, (2) threonine, (3) methionine and (4) isoleucine (hereinafter an agent for the prophylaxis or improvement of frailty containing at least isoleucine and threonine as active ingredients is sometimes to be generically abbreviated as the agent for the prophylaxis or improvement of frailty of the present invention).

While the amino acids of the above-mentioned (1) to (4) to be used in the present invention may be any of L-form, D-form and DL-form, the L-form is preferable.

The amino acids (1) to (4) may be salts, and examples of the salt of the amino acid include acid addition salt, metal salt, ammonium salt, organic amine addition salt, amino acid addition salt and the like.

Examples of the acid addition salt include inorganic acid salts such as hydrochloride, sulfate, nitrate, phosphate and the like, and organic acid salts such as acetate, maleate, fumarate, citrate, malate, lactate, α-ketoglutarate, gluconate, caprylate and the like.

Examples of the metal salt include alkali metal salts such as sodium salt, potassium salt and the like, alkaline earth metal salts such as magnesium salt, calcium salt and the like, aluminum salt, zinc salt and the like.

Examples of the ammonium salt include salts of ammonium, tetramethylammonium and the like.

Examples of the organic amine addition salt include salts with morpholine, piperidine and the like.

Examples of the amino acid addition salt include salts with glycine, phenylalanine, lysine, aspartic acid, glutamic acid and the like.

The salts of the amino acids (1) to (4) may be a hydrate (hydrate salt), and examples of such hydrate include 1 to 6 hydrates and the like.

The amino acids (1) to (4) in the present invention may be produced by any production method such as a protein hydrolysis method, a chemical synthesis method, an enzyme method, a fermentation method and the like, and commercially available products can also be used.

The amino acids (1) to (4) in the present invention can also be obtained by enzymatically hydrolyzing a natural protein having a sequence containing the amino acid.

In the present invention, “frailty” is one of the elderly person syndromes among the symptoms that appear along with aging, which is said to represent various physical and mental symptoms not necessarily considered illness. It refers to a “condition before developing health problems (life dysfunction, etc.), which is generated by a variety of factors involved in elderly generation that reduce the spare capacity of various organs playing an important role in life activity, and reduce the ability to adapt to changes in not only the internal environment of the body but also external environment, as a result of which physical, mental, social functions are gradually lost”.

Examples of the index or specific physical symptoms of frailty include body weight decrease, loss of muscle mass (shrinkage of body), decline in grip strength (faintness), feeling of fatigue (fatiguability), decrease in walking speed (slow movement), reduced amount of physical activity (less movement), in elderly person. When at least one of these symptoms is found, frailty is judged to be present. Among others, loss of muscle mass, decline in grip strength, feeling of fatigue, decrease in walking speed and decrease in the amount of physical activity, in elderly person are preferable targets of the agent for the prophylaxis or improvement of frailty of the present invention.

Specific examples of the mental symptoms of frailty include loss of motivation, depressed state, delirium, dementia, sleep disorder, anxiety disorder, social withdrawal and the like, in elderly person. Of these, loss of motivation, depressed state, delirium, dementia, anxiety disorder, and social withdrawal, in elderly person, are preferable targets of the agent for the prophylaxis or improvement of frailty of the present invention.

Prophylaxis of frailty is previous prevention of the above-mentioned symptoms from being developed and/or delaying the onset and/or progress of one of the above-mentioned symptoms, and improvement of frailty is a concept including bringing the above-mentioned symptoms to fall within a normal range, as well as preventing the progression (exacerbation) of the disease. The normal range here is determined based on the age, sex, height, basal metabolism, amount of motor activity and the like.

Examples of the cause of frailty include physical factors such as low nutrition, sarcopenia, contraction of a chronic disease, chronic inflammation, smoking, decrease in sex hormone and the like, social factors and environmental factors. Among these, the agent for the prophylaxis or improvement of frailty of the present invention is preferably used for frailty involving sarcopenia wherein muscle fiber number and muscle cross-sectional area decrease due to aging and physical functions are impaired by the overall loss of muscle mass.

The agent for the prophylaxis or improvement of frailty of the present invention can be applied to human, animals other than human, for example, mammals other than human (domestic animals and pet animals such as swine, bovine, horse, dog and the like), birds (poultry and pet animals such as turkey, chicken and the like) etc., and the like in elderly generation.

The elderly generation for human means 65 to around 74 years old (early-stage elderly generation) and after 75 years old (late-stage elderly generation). In addition, the World Health Organization (WHO) in the United Nations defines the elderly person as those of age 65 or above.

The agent for the prophylaxis or improvement of frailty of the present invention means pharmaceutical products, quasi-drugs, health aid foods, foods with health claims, compositions similar to pharmaceutical products and having a particular function and ingested for the purpose of maintaining health and the like such as supplement and the like, and functional foods, which are used for the “prophylaxis or improvement of frailty” in elderly person.

In the agent for the prophylaxis or improvement of frailty of the present invention, the weight ratio of isoleucine and threonine is generally 1:0.2 to 10, preferably 1:0.3 to 8, more preferably 1:0.5 to 5.

When tryptophan is contained, the weight ratio of isoleucine, threonine and tryptophan is generally 1:0.2 to 10:0.06 to 8, preferably 1:0.3 to 8:0.1 to 5, more preferably 1:0.5 to 5:0.2 to 3.

When methionine is contained, the weight ratio of isoleucine, threonine and methionine is generally 1:0.2 to 10:0.1 to 5, preferably 1:0.3 to 8:0.2 to 4, more preferably 1:0.5 to 5:0.3 to 3.

In the agent for the prophylaxis or improvement of frailty of the present invention, the weight ratio of tryptophan, threonine, methionine and isoleucine is generally 1:0.5 to 12:0.2 to 10:0.5 to 12, preferably 1:1 to 9:0.5 to 8:1 to 9, more preferably 1:2 to 6:1 to 5:2 to 6.

The weight (%) of isoleucine or threonine in the free amino acid in the agent for the prophylaxis or improvement of frailty of the present invention is generally not less than 3%, preferably not less than 10%, more preferably not less than 20%, particularly preferably not less than 30%, based on the total weight of the free amino acids in the agent. Generally, that of isoleucine or threonine is not more than 97%, preferably not more than 90%, more preferably not more than 80%, particularly preferably not more than 75%, especially preferably not more than 70%, based on the total weight of the free amino acids in the agent.

When tryptophan is contained, the weight (%) of each amino acid in the free amino acid in the agent for the prophylaxis or improvement of frailty of the present invention is generally isoleucine 2.5% to 80%, threonine 2.5% to 80% and tryptophan 0.8% to 50%, preferably 8% to 75%, 8% to 75% and 3.5% to 40%, more preferably 20% to 70%, 20% to 70% and 5% to 33%, particularly preferably 30% to 70%, 30% to 70% and 12% to 33%, based on the total weight of the free amino acids in the agent.

When methionine is contained, the weight (%) of each amino acid in the free amino acid in the agent for the prophylaxis or improvement of frailty of the present invention is generally isoleucine 2.5% to 80%, threonine 2.5% to 80% and methionine 1.6% to 60%, preferably 8% to 75%, 8% to 75% and 6% to 50%, more preferably 20% to 70%, 20% to 70% and 15% to 45%, particularly preferably 30% to 70%, 30% to 70% and 22% to 45%, based on the total weight of the free amino acids in the agent.

When 4 kinds of amino acids are contained, the weight (%) of each amino acid in the contained amino acid (free amino acid) in the agent for the prophylaxis or improvement of frailty of the present invention is generally (1) tryptophan 0.5% to 45%, (2) threonine 1.5% to 70%, (3) methionine 1.0% to 50%, (4) isoleucine 1.5% to 70%, preferably (1) 3.5% to 40%, (2) 5% to 65%, (3) 6% to 45%, (4) 5% to 65%, more preferably (1) 5% to 33%, (2) 8% to 60%, (3) 10% to 40%, (4) 8% to 60%, further preferably (1) 10% to 33%, (2) 20% to 60%, (3) 15% to 40%, (4) 20% to 60%, particularly preferably (1) 15% to 33%, (2) 25% to 60%, (3) 22% to 40%, (4) 25% to 60%, based on the total weight of the free amino acids in the agent.

The contained amino acid means a free amino acid, and does not include constituent amino acids in a protein or peptide.

While the dose of the agent for the prophylaxis or improvement of frailty of the present invention may vary depending on the age, sex, body weight, target disease, symptom, and dosage form, the daily dose of isoleucine and threonine in total is generally 40 mg to 15 g, preferably 67 mg to 13 g, more preferably 330 mg to 10 g, further preferably 330 mg to 4 g, particularly preferably 330 mg to 1.5 g, for an adult (e.g., body weight 60 kg), which is administered in once to several portions per day.

While the dose of the agent for the prophylaxis or improvement of frailty of the present invention may vary depending on the age, sex, body weight, target disease, symptom, and dosage form, the daily dose of isoleucine, threonine and tryptophan in total is generally 50 mg to 18 g, preferably 80 mg to 15 g, more preferably 400 mg to 12 g, further preferably 400 mg to 4 g, particularly preferably 400 mg to 1.6 g, for an adult (e.g., body weight 60 kg), which is administered in once to several portions per day.

While the dose of the agent for the prophylaxis or improvement of frailty of the present invention may vary depending on the age, sex, body weight, target disease, symptom, and dosage form, the daily dose of isoleucine, threonine and methionine in total is generally 50 mg to 20 g, preferably 90 mg to 18 g, more preferably 450 mg to 13 g, further preferably 450 mg to 4.5 g, particularly preferably 450 mg to 1.8 g, for an adult (e.g., body weight 60 kg), which is administered in once to several portions per day.

While the dose of the agent for the prophylaxis or improvement of frailty of the present invention may vary depending on the age, sex, body weight, target disease, symptom, and dosage form, the daily dose of amino acids (1) to (4) in total is generally 60 mg to 23 g, preferably 100 mg to 20 g, more preferably 500 mg to 15 g, further preferably 500 mg to 5 g, particularly preferably 500 mg to 2 g, for an adult (e.g., body weight 60 kg), which is administered in once to several portions per day.

The dose of (1) is generally 10 mg to 9 g, preferably 20 mg to 6 g, more preferably 80 mg to 5 g, particularly preferably 90 mg to 1 g, for an adult per day.

The dose of (2) is generally 30 mg to 20 g, preferably 60 mg to 18 g, more preferably 250 mg to 15 g, particularly preferably 250 mg to 1 g, for an adult per day.

The dose of (3) is generally 20 mg to 18 g, preferably 40 mg to 15 g, more preferably 170 mg to 12 g, particularly preferably 180 mg to 1 g, for an adult per day.

The dose of (4) is generally 30 mg to 20 g, preferably 60 mg to 18 g, more preferably 250 mg to 15 g, particularly preferably 250 mg to 1 g, for an adult per day.

The above-mentioned dose for an adult per day can be changed as appropriate in consideration of the sex, age, condition of the body such as disease and the like.

The above-mentioned dose of the agent for the prophylaxis or improvement of frailty of the present invention can be administered all at once or in several portions. The dosing period is not particularly limited, and long-term administration is possible since the components are derived from amino acids.

The dosage form of the agent for the prophylaxis or improvement of frailty of the present invention is not particularly limited and either of oral preparation or parenteral preparation can be employed. Examples of the dosage form thereof include agents for oral administration such as tablet, granule, powder, capsule, elixir, syrup, microcapsule, drink, emulsion, suspension and the like, skin external preparations such as ointment, cream, gel, liquid, lotion, facial mask, bathing powder and the like, injection and the like.

For oral administration, the agent for the prophylaxis or improvement of frailty of the present invention can contain, where necessary, a carrier, excipient, binder, swelling agent, lubricant, sweetening agent, flavor, preservative, emulsifier, coating agent and the like, and can be used together with these in a unit dosage form requested for the generally-acknowledged formulation and implementation. The amount of amino acid in these compositions or preparations only needs to be such amount that affords a suitable dose in the indicated range. In addition, the oral administration may be given any time before, after or between meals.

Examples of specific components that can be contained in the agent for the prophylaxis or improvement of frailty of the present invention include binders such as tragacanth, gum arabic, cornstarch and gelatin, polymeric polyvinylpyrrolidone and the like; excipients such as cellulose (e.g., microcrystalline cellulose, crystalline cellulose, hydroxypropylcellulose and the like) and a derivative thereof;

swelling agents such as cornstarch, pregelatinized starch, alginic acid, dextrin; lubricants such as magnesium stearate; flowability improvement agents such as fine silicon dioxide, methylcellulose; lubricants such as glycerol acid ester, talc, polyethylene glycol 6000 and the like; thickeners such as sodium carboxymethylcellulose, carboxyvinyl polymer, xanthan gum, gelatin and the like; sweetening agents such as sucrose, lactose and aspartam; flavors such as peppermint, vanilla flavor and cherry or orange; emulsifiers such as monoglyceride, polyglycerol ester of fatty acid, sucrose ester of fatty acid, lecithin, polyoxyethylenehydrogenated castor oil, polyoxyethylenemonostearic acid ester and the like; pH adjusters such as citric acid, sodium citrate, acetic acid, sodium acetate, sodium hydroxide and the like; flavoring agents such as aspartame, licorice extract, saccharin and the like; antioxidants such as erythorbic acid, butylhydroxyanisole, propyl gallate and the like; preservatives such as sodium benzoate, sodium edetate, sorbic acid, sodium sorbate, methyl parahydroxybenzoate, butyl p-hydroxybenzoate and the like, colorants such as red iron oxide, yellow ferric oxide, black ferric oxide, carmine, Food Color Blue No. 1, Food Color Yellow No. 4, Food Color Red No. 2 and the like; and the like.

When the formulation unit form is a capsule, the above-mentioned types of materials can further contain a liquid carrier such as fats and oils.

Also, various other materials can be added to change the physical form of a formulation unit. Examples of the coating agent for tablet include shellac, sugar or both and the like. Syrup or elixir can contain, for example, sucrose as a sweetening agent, methylparaben and propylparaben as preservative, dye and cherry or orange aroma and the like, and the like. Besides these, various vitamins and various amino acids may be added.

To provide an enteric preparation, for example, an aqueous solution of hydroxylphenylmethylcellulose is used as a coating pre-treatment agent, an aqueous solution of hydroxypropylmethylcellulosephthalate and an aqueous solution of polyacetin are used as coating agents and an enteric preparation is produced by a conventional method.

While the agent for the prophylaxis or improvement of frailty of the present invention contains at least isoleucine and threonine as active ingredients, an embodiment free of amino acids other than isoleucine and threonine, preferably, an embodiment free of amino acids other than isoleucine, threonine and tryptophan, or an embodiment free of amino acids other than isoleucine, threonine and methionine, can be mentioned, and an embodiment substantially free of amino acids other than (1) to (4) is more preferable. The term “substantially free” means that the agent contains less than 0.2 wt %, preferably less than 20 0.1 wt %, even more preferably less than 0.05 wt %, of a peptide or protein, based on the total weight of the agent.

The agent for the prophylaxis or improvement of frailty of the present invention may further contain any other active ingredients and other amino acids. As other amino acid, essential amino acid can be mentioned, and valine is preferable. The content of other amino acid is preferably not more than 60 wt %, more preferably not more than 40 wt %, further preferably not more than 20 wt %, of the amino acid contained.

The agent for the prophylaxis or improvement of frailty of the present invention may be used concurrently with other proteins and peptides. The content of protein and peptide is preferably not more than 90 wt %, more preferably not more than 80 wt %, in consideration of a burden on the kidney of elderly person, and an embodiment substantially free of protein and peptide may also be used. The term “substantially free” means that the agent contains less than 0.2 wt %, preferably less than 0.1 wt %, even more preferably less than 0.05 wt %, of a peptide or protein, based on the total weight of the agent.

For parenteral administration, for example, a solution containing amino acids (1) to (4) can be nasally sprayed, administered as an injection and the like. When the agent for the prophylaxis or improvement of frailty of the present invention is used as a skin external preparation, amino acids (1) to (4) are dispersed in various bases, additives are added and the mixture is formulated according to a conventional method. Examples of such base and additive include higher fatty acid esters such as petrolatum, liquid paraffin, myristic acid isopropyl, myristic acid octyldodecyl and the like, higher alcohols such as squalane, lanolin, cetanol and the like, fats and oils-base such as silicone oil, fats and oils from plant or animal and the like, lower alcohols such as ethanol and the like, polyhydric alcohols such as polyethylene glycol, propylene glycol and the like, emulsion or emulsion stabilizer such as α-monoglycerylether, lecithin, sorbitan ester of fatty acid, dextrin fatty acid ester, fatty acid monoglyceride, fatty acid metal salt, magnesium sulfate and the like, aromatic, preservative, dye, thickener, antioxidant, UV protective agent, various medicinal agents such as wound healing agent, anti-inflammatory agent, moisturizer and the like, water and the like.

In the present invention, a pharmaceutical product may be the agent for the prophylaxis or improvement of frailty of the present invention itself, or may contain other additives and the like.

When the agent for the prophylaxis or improvement of frailty of the present invention is used as a food, it means a health food ingested by taking note of the particular function of the present invention, as well as a food for specified health uses and a food with nutrient function claims defined in the food with health claims, and also encompasses dietary supplements. The amount of amino acid contained in the food, which is eaten or drank per day, preferably falls within the same range mentioned above as that in the agent for the prophylaxis or improvement of frailty of the present invention. The form of a food with health claims as the agent for the prophylaxis or improvement of frailty of the present invention is not particularly limited.

While the food includes general food forms, for example, the agent for the prophylaxis or improvement of frailty of the present invention, and soup such as miso soup, corn soup and the like, drinks such as green tea, coffee, tea, beverage and the like, powder drinks, confectionery such as chocolate, cookies and the like, ice cream, jelly and the like, which contain the agent and a suitable flavor, can be mentioned.

As the food in the present invention, a package form of a unit ingestion amount per serving of amino acid selected from (1) to (4) including at least isoleucine and threonine, a form of a drink of a suspension or solution of the aforementioned amino acid, which is filled in a bottle and the like for a single serving and the like can be mentioned. The dose per serving may be the above-mentioned daily dose.

Specifically, an agent for the prophylaxis or improvement of frailty in a unit package form per serving, which contains, in the unit, generally 4 mg to 15 g, preferably 7 mg to 13 g, further preferably 33 mg to 10 g, particularly preferably 33 mg to 4 g, of a total amount of isoleucine and threonine as an ingestion amount per serving can be mentioned. Particularly, an agent for the prophylaxis or improvement of frailty containing 33 mg to 1.5 g is preferable.

In addition, an agent for the prophylaxis or improvement of frailty in a unit package form per serving, which contains, in the unit, generally 5 mg to 18 g, preferably 8 mg to 15 g, further preferably 40 mg to 12 g, particularly preferably 40 mg to 4 g, of a total amount of isoleucine, threonine and tryptophan as an ingestion amount per serving can be mentioned. Particularly, an agent for the prophylaxis or improvement of frailty containing 40 mg to 1.6 g is preferable.

Furthermore, an agent for the prophylaxis or improvement of frailty in a unit package form per serving, which contains, in the unit, generally 5 mg to 20 g, preferably 9 mg to 18 g, further preferably 45 mg to 13 g, particularly preferably 45 mg to 4.5 g, of a total amount of isoleucine, threonine and methionine as an ingestion amount per serving can be mentioned. Particularly, an agent for the prophylaxis or improvement of frailty containing 45 mg to 1.8 g is preferable.

In addition, an agent for the prophylaxis or improvement of frailty in a unit package form per serving, which contains, in the unit, generally 6 mg to 23 g, preferably 10 mg to 20 g, further preferably 50 mg to 15 g, particularly preferably 60 mg to 10 g, of a total amount of amino acids (1) to (4) as an ingestion amount per serving can be mentioned. Particularly, an agent for the prophylaxis or improvement of frailty containing 60 mg to 2 g is preferable.

In addition, a food or drink for the prophylaxis or improvement of frailty in a unit package form per serving, which contains, in the unit, not less than 0.04 g of isoleucine and threonine in total, wherein the weight ratio of isoleucine and threonine is 1:0.2 to 10 is also one embodiment of the present invention. The above-mentioned total amount is preferably not less than 0.08 g, and preferably not less than 0.2 g. It is generally not more than 20 g, preferably not more than 12 g, more preferably not more than 8 g. The food and drink are similarly defined as the above-mentioned food.

Similarly, a food or drink for the prophylaxis or improvement of frailty in a unit package form per serving, which contains, in the unit, not less than 0.06 g of 4 kinds of amino acids of tryptophan, threonine, methionine and isoleucine in total, wherein the weight ratio of tryptophan, threonine, methionine and isoleucine is 1:0.5 to 12:0.2 to 10:0.5 to 12 is also one embodiment of the present invention. The above-mentioned total amount is preferably not less than 0.12 g, and preferably not less than 0.3 g. It is generally not more than 25 g, preferably not more than 18 g, more preferably not more than 12 g. The food and drink are similarly defined as the above-mentioned food.

Since a small amount of the aforementioned food or drink for the prophylaxis or improvement of frailty is effective, it is preferable for a dosage form such as tablet, granule, capsule and the like, and can be conveniently ingested.

More concretely, a unit package form per serving, which contains, as an ingestion amount per serving in the unit, generally (1) 1 mg to 9 g, (2) 3 mg to 20 g, (3) 2 mg to 18 g, (4) 3 mg to 20 g, preferably (1) 2 mg to 6 g, (2) 6 mg to 18 g, (3) 4 mg to 15 g, (4) 6 mg to 18 g, more preferably (1) 8 mg to 5 g, (2) 25 mg to 15 g, (3) 17 mg to 12 g, (4) 25 mg to 15 g, of amino acids (1) to (4), can be mentioned. Therefore, when isoleucine and threonine are contained, and tryptophan and/or methionine are/is further contained, the above-mentioned amounts can be contained in combination in the unit package form.

In the case of a unit package form of 10 to 60 servings, the above-mentioned numerical values can be set to 10- to 60-fold.

In consideration of a food containing other amino acid(s), the weight (%) of each amino acid in the contained amino acids in the food or drink containing amino acids (1) to (4) of the present invention is (1) tryptophan 0.5% to 45%, (2) threonine 1.5% to 70%, (3) methionine 1.0% to 50%, (4) isoleucine 1.5% to 70%, preferably (1) 3.5% to 40%, (2) 5% to 65%, (3) 6% to45%, (4) 5% to 65%, further preferably (1) 5% to 33%, (2) 8% to 60%, (3) 10% to 40%, (4) 8% to 60%.

Specifically, a food or drink containing preferably not more than 60 wt %, more preferably not more than 40 wt %, further preferably not more than 20 wt %, of amino acid other than (1) to (4) of the contained amino acid is preferable.

Another embodiment of the present invention is a kit containing a measuring container, and a food or drink for the prophylaxis or improvement of frailty containing isoleucine and threonine as active ingredients.

The measuring container is not particularly limited as long as it is a container for measuring the amount of single use of the above-mentioned amino acids and, for example, a measuring cup, a measuring spoon and the like can be mentioned. The amount of single use is the same as the amount described in the above-mentioned unit package form per serving and, for example, a total amount of not less than 0.04 g of isoleucine and threonine can be mentioned. The amount that can be measured by a measuring container can be determined according to the container such as a level amount, a heaping amount and the like. The measuring container may have a scale showing the amount of single use and the like.

Another embodiment of the present invention is a kit containing a measuring container, and a food or drink for the prophylaxis or improvement of frailty containing 4 kinds of amino acids of tryptophan, threonine, methionine, and isoleucine as active ingredients.

The measuring container is not particularly limited as long as it is a container for measuring the amount of single use of the above-mentioned amino acids and, for example, a measuring cup, a measuring spoon and the like can be mentioned.

The amount of single use is the same as the amount described in the above-mentioned unit package form per serving and, for example, a total amount of not less than 0.06 g of the 4 kinds of amino acids can be mentioned. The amount that can be measured by a measuring container can be determined according to the container such as a level amount, a heaping amount and the like. The measuring container may have a scale showing the amount of single use and the like.

Other features of the invention will become apparent in the course of the following descriptions of exemplary embodiments which are given for illustration of the invention and are not intended to be limiting thereof.

### EXAMPLES

In the present specification, % indicates wt % unless particularly indicated.

### Example 1

### Granular Powdered Nutrition Composition

In consideration of the effect on the prophylaxis of frailty, a granular powder nutrition composition was prepared based on the composition shown in Table 1. That is, a mixture having the composition of Table 1 was mixed in a compact high-speed mixer (NSK-150S, manufactured by Okadaseiko. co. jp) for 5 minutes. To the mixture were added distilled water and 99.5% alcohol at 2 to 5 wt %, and the mixture was kneaded in the mixer for 5 minutes to give a moistened kneaded mixture. The moistened kneaded mixture was granulated by an extrusion granulating machine with a 1.0 mm₉ screen, the obtained form was dried at a normal pressure and 70° C. for 2 hours, sieved and 1.05 g of granules was filled in an aluminum bag. The whole components of the granular powder nutrition composition were stably maintained even one year later. In addition, the granular powder nutrition composition could be used by mixing with a nutritional supplement dissolved in warm water, and could be formed as an ion drink. Furthermore, it could be further mixed with a spice, salts such as sodium chloride and the like, sodium glutamate, nucleic acid and the like, and added to various foods.

TABLE 1

| | unit | wt % (weight ratio) |
|---|---|---|
| L-tryptophan | % | 10.46 (1.0) |
| L-threonine | % | 30.70 (2.9) |
| L-methionine | % | 21.01 (2.0) |
| L-isoleucine | % | 32.89 (3.1) |
| sucrose ester of fatty acid | % | 2.85 |
| flavor preparation | % | 0.95 |
| lecithin | % | 1.14 |
| 99.5% alcohol | | — |
| distilled water | | — |
| total | % | 100.0 |

### Example 2

### Granular Powdered Nutrition Composition

In the same manner as in Example 1 except that a mortar was used for kneading instead of the high-speed mixer, and a horizontal extruder (manufactured by Umetani Tekkosho) was used as the extrusion granulating machine, a granular powder could also be obtained.

### Example 3

### Jelly Nutrition Composition

In consideration of the effect on the prophylaxis of frailty, a jelly nutrition composition was prepared as a nutrition composition based on the composition shown in Table 2. That is, to the composition of Table 2 were added a polysaccharide thickener and water, mixed, and emulsified by stirring with heating. After cooling, pH was adjusted to 3.8, and the mixture was sterilized by heating at 80° C. for 10 minutes, 1.25 g was cooled and filled in a pouch. The whole components of the jelly nutrition composition were stably maintained even one year later.

TABLE 2

| | unit | wt % (weight ratio) | wt % (weight ratio) |
|---|---|---|---|
| L-tryptophan | % | 10 (1.0) | 10 (1.0) |
| L-threonine | % | 30 (3.0) | 41 (4.1) |
| L-methionine | % | 19 (1.9) | 10 (1.0) |
| L-isoleucine | % | 22 (2.2) | 20 (2.0) |
| lactose | % | 12 | 12 |
| sucrose | % | 7 | 7 |
| total | % | 100 | 100 |

### Example 4

### Favorite Drink Powder

In careful consideration of the effect on the prophylaxis of frailty, powder preference drinks were prepared. The amounts of the starting materials to realize them are shown in Tables 3 to 6. Mixtures having the compositions of respective Tables were mixed by a high-speed mixer, and filled in aluminum bags. By changing the powder, flavor and the like, Caramel Macchiato, café mocha, cocoa, powdered green tea au lait and the like could be produced similarly.

TABLE 3
Coffee

| | unit | wt % (weight ratio) |
|---|---|---|
| L-tryptophan | % | 0.50 (1.0) |
| L-threonine | % | 1.60 (3.2) |
| L-methionine | % | 1.05 (2.1) |
| L-isoleucine | % | 1.60 (3.2) |
| sucrose | % | 41.55 |
| coffee whitener | % | 42.12 |
| coffee powder | % | 11.58 |
| total | % | 100 |

TABLE 4
Café au lait

| | unit | wt % (weight ratio) |
|---|---|---|
| L-tryptophan | % | 0.34 (1.0) |
| L-threonine | % | 1.03 (3.0) |
| L-methionine | % | 0.68 (2.0) |
| L-isoleucine | % | 1.00 (2.9) |
| sucrose | % | 35.49 |
| coffee whitener | % | 23.27 |
| coffee powder | % | 5.82 |
| whey powder | % | 23.27 |
| defatted milk powder | % | 7.27 |
| sodium chloride | % | 0.29 |
| caramel dye | % | 0.39 |
| milk flavor | % | 0.62 |
| coffee flavor | % | 0.48 |
| xanthan gum | % | 0.05 |
| total | % | 100 |

TABLE 5
Green tea

| | unit | wt % (weight ratio) |
|---|---|---|
| L-tryptophan | % | 5.05 (1.0) |
| L-threonine | % | 15.15 (3.0) |
| L-methionine | % | 10.10 (2.0) |
| L-isoleucine | % | 15.15 (3.0) |
| green tea powder | % | 16.36 |
| vitamin C | % | 1.82 |
| sodium hydrogen carbonate | % | 1.82 |
| maltodextrin | % | 25.64 |
| green tea flavor | % | 1.64 |
| 0.25% aspartame | % | 7.27 |
| total | % | 100 |

TABLE 6
Milk tea

| | unit | wt % (weight ratio) |
|---|---|---|
| L-tryptophan | % | 1.00 (1.0) |
| L-threonine | % | 2.00 (2.0) |
| L-methionine | % | 1.00 (1.0) |
| L-isoleucine | % | 2.00 (2.0) |
| sucrose | % | 61.75 |
| coffee whitener | % | 8.38 |
| tea powder | % | 1.61 |
| whey powder | % | 20.93 |
| sodium ascorbate | % | 0.63 |
| sucrose fatty acid ester | % | 0.31 |
| aspartame | % | 0.03 |
| milk flavor | % | 0.02 |
| tea flavor | % | 0.14 |
| emulsifier | % | 0.20 |
| total | % | 100 |

### Example 5

### Powdered Soup

In consideration of the effect on the prophylaxis of frailty, powdered soups were made based on the compositions shown in Tables 7 to 10. The starting materials shown in s respective Tables were mixed by a high-speed mixer, and granulated in a fluidized bed. Furthermore, consomme soup and pumpkin soup could be produced similarly.

TABLE 7
Onion soup

| | unit | wt % (weight ratio) |
|---|---|---|
| L-tryptophan | % | 2.00 (1.0) |
| L-threonine | % | 4.00 (2.0) |
| L-methionine | % | 3.00 (1.5) |
| L-isoleucine | % | 4.00 (2.0) |
| glucose | % | 20.75 |
| sucrose | % | 17.62 |
| sodium chloride | % | 25.87 |
| onion powder | % | 7.16 |
| garlic powder | % | 0.25 |
| potato starch | % | 7.34 |
| natural seasoning | % | 3.67 |
| white pepper powder | % | 0.21 |
| caramel dye | % | 0.37 |
| citric acid | % | 0.22 |
| consommé | % | 0.60 |
| umami seasoning | % | 2.93 |
| aspartame | % | 0.01 |
| total | % | 100 |

TABLE 8
Chinese soup

| | unit | wt % (weight ratio) |
|---|---|---|
| L-tryptophan | % | 3.00 (1.0) |
| L-threonine | % | 9.00 (3.0) |
| L-methionine | % | 6.00 (2.0) |
| L-isoleucine | % | 7.00 (2.3) |
| Chicken powder | % | 2.75 |
| chicken stock soup | % | 1.50 |
| chicken oil | % | 1.13 |
| sucrose | % | 6.75 |
| sodium chloride | % | 28.50 |
| natural seasoning | % | 0.94 |
| umami seasoning | % | 15.60 |
| maltodextrin | % | 16.04 |
| chicken flavor | % | 1.58 |
| turmeric | % | 0.15 |
| white pepper powder | % | 0.03 |
| ginger powder | % | 0.03 |
| total | % | 100 |

TABLE 9
Corn soup

| | unit | wt % (weight ratio) |
|---|---|---|
| L-tryptophan | % | 0.50 (1.0) |
| L-threonine | % | 1.00 (2.0) |
| L-methionine | % | 0.80 (1.6) |
| L-isoleucine | % | 1.00 (2.0) |
| Corn powder | % | 17.04 |
| cheese | % | 1.26 |
| powder cream | % | 16.85 |
| potato starch | % | 10.00 |
| sucrose | % | 22.70 |
| sodium chloride | % | 5.10 |
| onion powder | % | 5.26 |
| palm oil | % | 5.20 |
| whole milk powder | % | 5.02 |
| condensed milk | % | 3.80 |
| maltodextrin | % | 1.24 |
| guar gum | % | 1.05 |
| natural seasoning | % | 0.52 |
| umami seasoning | % | 0.32 |
| corn flavor | % | 1.20 |
| β-carotene | % | 0.08 |
| turmeric | % | 0.04 |
| aspartame | % | 0.02 |
| total | % | 100 |

TABLE 10
Miso soup

| | unit | wt % (weight ratio) |
|---|---|---|
| L-tryptophan | % | 1.00 (1.0) |
| L-threonine | % | 3.30 (3.3) |
| L-methionine | % | 2.00 (2.0) |
| L-isoleucine | % | 3.00 (3.0) |
| miso powder | % | 57.91 |
| powder of dried bonito | % | 21.88 |
| bonito stock | % | 0.17 |
| sucrose | % | 3.45 |
| sodium chloride | % | 1.21 |
| natural seasoning | % | 1.79 |
| umami seasoning | % | 3.60 |
| citric acid | % | 0.69 |
| total | % | 100 |

### Example 6

### Chocolate

In consideration of the effect on the prophylaxis of frailty, chocolate was prepared based on the composition shown in Table 11. Chocolate was dissolved in a water bath, and a flavor and amino acid were added and mixed well. The mixture was flown in a mold, and solidified by cooling in a refrigerator.

TABLE 11

| | unit | wt % (weight ratio) |
|---|---|---|
| L-tryptophan | % | 0.50 (1.0) |
| L-threonine | % | 2.00 (4.0) |
| L-methionine | % | 1.00 (2.0) |
| L-isoleucine | % | 1.50 (3.0) |
| black chocolate | % | 72.96 |
| white chocolate | % | 21.89 |
| chocolate flavor | % | 0.15 |
| total | % | 100 |

### Example 7

### Cookie

In consideration of the effect on frailty, a cookie was prepared based on the composition shown in Table 12. Sucrose was added to butter softened at room temperature, stirred well, and an egg was added and mixed. The remaining powder starting materials were added, and the mixture was stirred in a mixer, formed with a squeezer and baked in an oven at 165° C. for 18 minutes.

TABLE 12

| | unit | wt % (weight ratio) |
|---|---|---|
| L-tryptophan | % | 0.20 (1.0) |
| L-threonine | % | 1.00 (5.0) |
| L-methionine | % | 0.50 (2.5) |
| L-isoleucine | % | 0.80 (4.0) |
| butter | % | 30.08 |
| sucrose | % | 16.50 |
| egg | % | 13.72 |
| sodium chloride | % | 0.26 |
| whole milk powder | % | 1.00 |
| soft wheat-flour | % | 35.90 |
| milk flavor | % | 0.04 |
| total | % | 100 |

### Experimental Example 1

### Consideration of Effect of Amino Acid administration on elderly person at home.

Based on the production method of Example 1, granules containing L-tryptophan, L-threonine, L-methionine and L-isoleucine at a weight ratio of 1:2.9:2.0:3.1 were produced. The granule corresponding to the total amount of 1 g of amino acid was ingested by 11 elderly persons (3 males, 8 females, average age 80.4±7.6) once per day for 2 months. Physical measurement was performed before and after the start of the amino acid granule ingestion, and grasping power, thigh circumference, walking speed and the like were measured.

In a seated position on a chair with both arms put down naturally on both sides of the body, a dynamometer (TTM yoken dynamo meter 50 kg YCII senior & child) was held in a hand. The grasping range was adjusted to insure easy holding, and the subjects were instructed to grip the dynamometer as tight as possible. Measurement was performed once for right and left each at 0.1 kg unit, and the values thereof were recorded. In the analysis, the right and left were compared, and the maximum value was taken as the dominant hand and used for the test.

For statistical processing, an analysis software JMP9 was used, and the t-test of the related 2 groups was performed. The data are shown in mean±S.D., significant level was *p<0.05, and critical rate was 5%.

The results of the physical measurement before and after the start of the amino acid granule ingestion are shown in Table 13. As a result of the comparison of before and after the start of the amino acid granule ingestion, the grip strength and walking speed were significantly improved after the amino acid granule ingestion. Furthermore, the thigh circumference was maintained or improved for 2 months in 9 out of 11 subjects.

TABLE 13
Results of physical measurement before and after
amino acid granule ingestion.

| | | 2 months after ingestion | | |
|---|---|---|---|---|
| | | | | number of |
| | before | | | subjects with |
| | ingestion | | | improvement, |
| | mean ± S.D. | mean ± S.D. | p value | maintenance |
| age (years old) | 80.4 ± 7.6 | — | — | |
| grip strength | 15.0 ± 4.1 | 17.9 ± 5.4 | 0.025* | 8/11 |
| (kg) | | | | |
| thigh | 34.4 ± 4.2 | 35.0 ± 3.5 | 0.199 | 9/11 |
| circumference | | | | |
| (cm) | | | | |
| walking speed | 35.0 ± 20.0 | 43.7 ± 23.8 | 0.036* | 11/11 |
| (m/min) | | | | |

As a result of interview search by asking “Do you have any difficulty or distress at the moment?”, 6 out of 11 subjects had some difficulty or distress before the start of the amino acid granule ingestion, whereas the number significantly decreased to 2 out of 11 subjects after the start of the amino acid granule ingestion.

### Experimental Example 2

### Consideration of Effect of 4 Kinds of Amino Acids on Low Protein Model Rat

### Test Method

### (1) Production of Low Protein Model

Using 10-week-old CD(SD) male rats, they were raised on a 1 wt % casein feed obtained by reducing the protein in standard purified diet AIN-93G (manufactured by Oriental yeast Co., Ltd.) (Table 14). The animal fed on the 1 wt % casein diet for 28 days (4 weeks) was used as a low protein model, and an influence of amino acid administration was evaluated.

### (2) Administration Schedule

10-week-old CD(SD) male rats were divided into the groups shown in Table 15, casein 20 wt % (20% C group) or 1 wt % feed was fed for 28 days and the same feed was given thereafter to the 20% C group according to the schedule shown in FIG. 1. The low protein model rats were continuously given a 1 wt % casein feed obtained by reducing the protein in standard purified diet AIN-93G (1% C group) or a feed added with each amino acid shown 5 in Table 16 (4AA group) for 2 weeks.

### (3) Measurement of Body Weight and Spontaneous Activity

A daily feed intake was measured every day by using a balance system (Shimadzu Corporation) from the day when the amino acid added diet was given.

Spontaneous activity was measured by Low-Cost & Multi-Channel System SUPERMEX (Muromachi Kikai. Co., Ltd.) utilizing a passive infrared sensor on the day before giving the amino acid added feed and in the dark period (12 hr) on day 12 from the feeding.

TABLE 14
Amount of amino acid (g) to be added per 1.0 kg feed
(casein 10 g)

| | |
|---|---|
| Ile | 0.399 |
| Met | 0.254 |
| Thr | 0.371 |
| Trp | 0.127 |
| total | 1.151 |
| % | 0.115 |

TABLE 15

| group constitution | casein | addition amino acid | n |
|---|---|---|---|
| 20% C | 20 wt % | — | 10 |
| 1% C | 1 wt % | — | 11 |
| 4AA | 1 wt % | Ile/Met/Thr/Trp | 11 |

TABLE 16
Composition table (%) of test diet

| | 20% | | | | | | | | |
|---|---|---|---|---|---|---|---|---|---|
| | casein diet | 1% casein diet | | | | | | | |
| | 20% C | 1% C | 4AA | -Trp | -Thr | -Met | Ile, Thr | Ile | Thr |
| protein | | | | | | | | | |
| milk casein | 20 | 1 | 1 | 1 | 1 | 1 | 1 | 1 | 1 |
| L-cystine | 0.3 | 0.3 | 0.3 | 0.3 | 0.3 | 0.3 | 0.3 | 0.3 | 0.3 |
| Ile | | | 0.040 | 0.040 | 0.040 | 0.040 | 0.040 | 0.040 | |
| Met | | | 0.025 | 0.025 | 0.025 | | | | |
| Thr | | | 0.037 | 0.037 | | 0.037 | 0.037 | | 0.037 |
| Trp | | | 0.013 | | 0.013 | 0.013 | | | |
| hydrocarbonate | | | | | | | | | |
| cornstarch | 39.57 | 56.75 | 56.75 | 56.75 | 56.75 | 56.75 | 56.75 | 56.75 | 56.75 |
| α-cornstarch | 13.2 | 13.2 | 13.2 | 13.2 | 13.2 | 13.2 | 13.2 | 13.2 | 13.2 |
| granulated sugar | 10 | 10 | 10 | 10 | 10 | 10 | 10 | 10 | 10 |
| TK16 | | 2 | 2 | 2 | 2 | 2 | 2 | 2 | 2 |
| lipid | | | | | | | | | |
| purified soybean | 7 | 7 | 7 | 7 | 7 | 7 | 7 | 7 | 7 |
| oil | | | | | | | | | |
| cellulose powder | 5 | 5 | 5 | 5 | 5 | 5 | 5 | 5 | 5 |
| AIN-93VX | 1 | 1 | 1 | 1 | 1 | 1 | 1 | 1 | 1 |
| AIN-93G-MX | 3.5 | 3.5 | 3.5 | 3.5 | 3.5 | 3.5 | 3.5 | 3.5 | 3.5 |
| choline | 0.25 | 0.25 | 0.25 | 0.25 | 0.25 | 0.25 | 0.25 | 0.25 | 0.25 |
| bitartrate | | | | | | | | | |
| tertiary | 0.0014 | 0.0014 | 0.0014 | 0.0014 | 0.0014 | 0.0014 | 0.0014 | 0.0014 | 0.0014 |
| butylhydroquinone | | | | | | | | | |
| total | 100 | 100 | 100 | 100 | 100 | 100 | 100 | 100 | 100 |

### Results

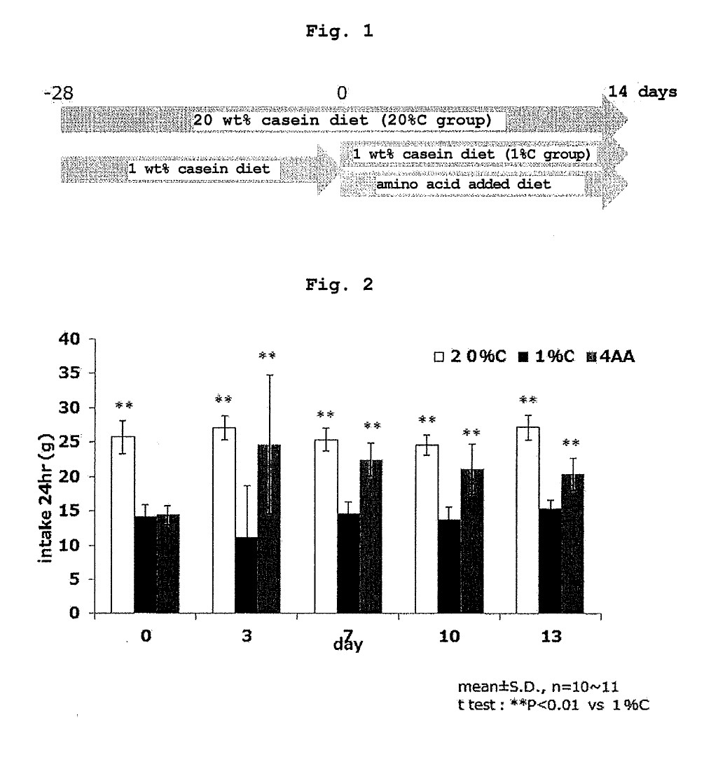

The feed intake is shown in FIG. 2. In the 4AA addition free group, the feed intake decreased, but it was significantly improved by giving 4AA.

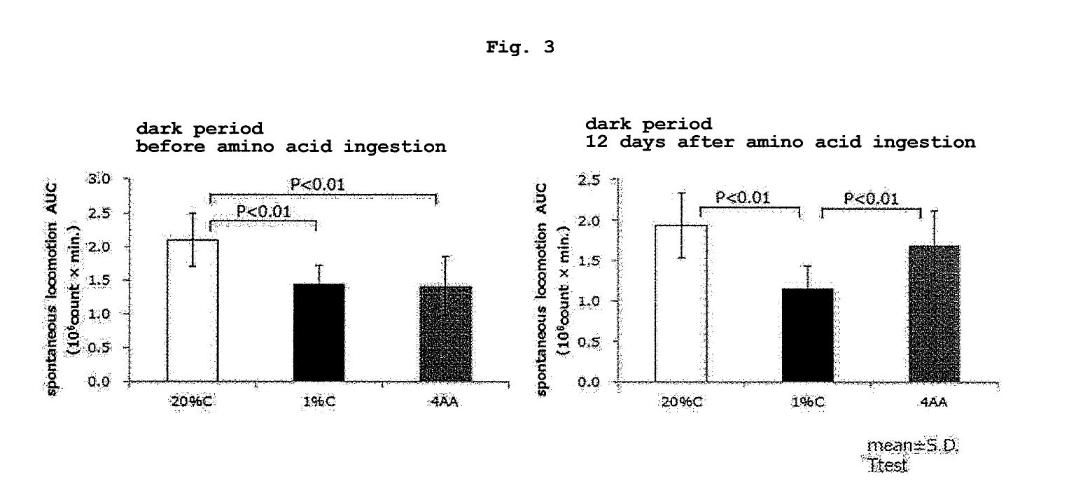

The spontaneous activity in the dark period significantly decreased in the 4AA addition free group compared to the control group, but it was significantly recovered by 4AA ingestion (remarkably observed particularly in the dark period). The results thereof are shown in FIG. 3.

### Experimental Example 3

### Consideration of Influence of Various Amino Acids on Low Protein Model Rat—1

### Test Method

10-week-old CD(SD) male rats were divided into the groups shown in Table 17, casein 20 wt % (20% C group) or 1 wt % feed was fed for 28 days according to the schedule shown in FIG. 1, and the body weight of rats thereafter fed with a 1 wt % casein diet (1% C group),and further added with each amino acid shown in Table 16 (4AA addition group (4AA group); a group of 4AA less tryptophan (−Trp group); a group of 4AA less methionine (−Met group); and a group of 4AA less threonine (−Thr group)) was measured.

TABLE 17

| group constitution | casein | amino acid added | n |
|---|---|---|---|
| 20% C | 20 wt % | — | 6 |
| 1% C | 1 wt % | — | 6 |
| 4AA | 1 wt % | Ile/Met/Thr/Trp | 6 |
| -Trp | 1 wt % | Ile/Met/Thr | 6 |
| -Thr | 1 wt % | Ile/Met/Trp | 6 |
| -Met | 1 wt % | Ile/Thr/Trp | 6 |

### Results

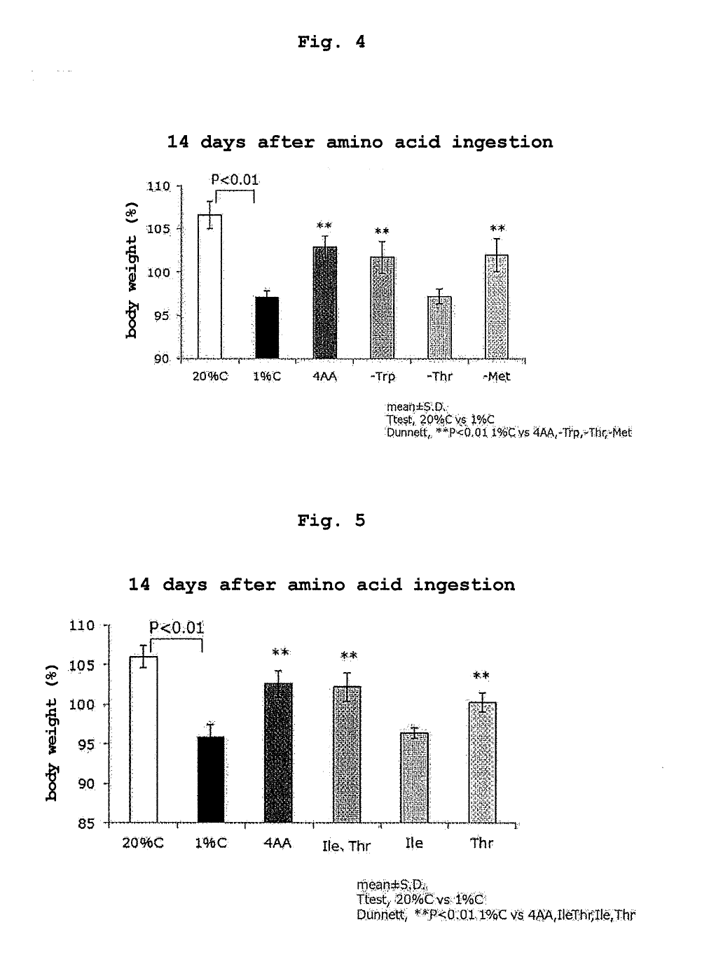

The body weight of the rats 14 days after feeding with the amino acid added diet was measured, the ratio (%) to the body weight measured on day 0 was calculated and shown in FIG. 4. In the 1% C group, the body weight decreased, whereas the body weight was significantly improved in the 4AA group, −Trp group and −Met group. In the −Thr group, the body weight was not improved.

### Experimental Example 4

### Consideration of Influence of Various Amino Acids on Low Protein Model Rat—2.

### Test Method

10-week-old CD(SD) male rats were divided into the groups shown in Table 18, casein 20 wt % (20% C group) or 1 wt % feed was fed for 28 days according to the schedule shown in FIG. 1, and the body weight of rats thereafter fed with a 1 wt % casein diet (1% C group), and further added with the amino acid shown in Table 16 (4AA addition group (4AA group); an isoleucine and threonine combined use group (Ile, Thr group); an isoleucine addition group (Ile group); and the a threonine addition group (Thr group)) was measured.

TABLE 18

| group | | | |
|---|---|---|---|
| constitution | casein | amino acid added | n |
| 20% C | 20 wt % | — | 6 |
| 1% C | 1 wt % | — | 6 |
| 4AA | 1 wt % | Ile/Met/Thr/Trp | 6 |
| Ile, Thr | 1 wt % | Ile/Thr | 6 |
| Ile | 1 wt % | Ile | 6 |
| Thr | 1 wt % | Thr | 6 |

### Results

The body weight of the rats 14 days after feeding with the amino acid added diet was measured, the ratio (%) to the body weight measured on day 0 was calculated and shown in FIG. 5. In the 1% C group, the body weight decreased, whereas the body weight was significantly recovered in the 4AA group, Ile, Thr group, and Thr group. In the Ile group, increase of the body weight was weakened.

### Experimental Example 5

### Consideration of Influence of Other Amino Acids on Low Protein Model Rat

### Test Method

10-week-old CD(SD) male rats were divided into the groups shown in Table 19, casein 20 wt % (20% C group) or 1 wt % feed was fed for 28 days according to the schedule shown in FIG. 1, and the body weight and weight of viscera-isolated carcass of rats thereafter fed for 14 days with a 1 wt % casein diet (1% C group), and further added with 1.15 g of the amino acid shown in Table 20 per 1 kg feed (4AA addition group (4AA group); a group of essential and non-essential amino acids addition group less 4AA (ALL-4AA group); essential amino acid addition group less 4AA (EAA-4AA group), non-essential amino acid addition group (NEAA group); and glycine addition group (Gly group)) was measured. The weight of viscera-isolated carcass to be the index of the weight of the skeletal muscle was obtained by measuring the body weight on day 14 from ingestion of each amino acid addition diet, exsanguinating the rat under anesthesia, isolating the viscera, and measuring the carcass.

TABLE 19

| group | | | |
|---|---|---|---|
| constitution | casein | amino acid added | n |
| 20% C | 20 wt % | — | 6 |
| 1% C | 1 wt % | — | 6 |
| 4AA | 1 wt % | Ile/Met/Thr/Trp | 6 |
| ALL-4AA | 1 wt % | ALL-4AA | 6 |
| EA-4AA | 1 wt % | EAA-4AA | 6 |
| NEAA | 1 wt % | NEAA | 6 |
| Gly | 1 wt % | Gly | 6 |

TABLE 20
Composition table (%) of test diet

| | | 20% | | | | | |
|---|---|---|---|---|---|---|---|
| | | casein diet | | | | | |
| | | control | no addition | ALL-4AA | EAA-4AA | NEAA | Gly |
| protein | milk casein | 20 | 1 | | | | |
| | L-cystine | 0.3 | 0.3 | | | | |
| | essential, non-essential | | | 0.115 | | | |
| | amino acid other than 4AA | | | | | | |
| | essential amino acid other | | | | 0.115 | | |
| | than 4AA | | | | | | |
| | non-essential amino acid | | | | | 0.115 | |
| | L-glycine | | | | | | 0.115 |
| hydrocarbonate | cornstarch | 39.75 | 56.75 | 56.75 | 56.75 | 56.75 | 56.75 |
| | α-cornstarch | 13.2 | 13.2 | 13.2 | 13.2 | 13.2 | 13.2 |
| | granulated sugar | 10 | 10 | 10 | 10 | 10 | 10 |
| | TK16 | | 2 | 2 | 2 | 2 | 2 |
| lipid | purified soybean oil | 7 | 7 | 7 | 7 | 7 | 7 |
| | cellulose powder | 5 | 5 | 5 | 5 | 5 | 5 |
| | AIN-93VX | 1 | 1 | 1 | 1 | 1 | 1 |
| | AIN-93G-MX | 3.5 | 3.5 | 3.5 | 3.5 | 3.5 | 3.5 |
| | choline bitartrate | 0.25 | 0.25 | 0.25 | 0.25 | 0.25 | 0.25 |
| | tertiary butylhydroquinone | 0.0014 | 0.0014 | 0.0014 | 0.0014 | 0.0014 | 0.0014 |
| | total | 100 | 100 | 100 | 100 | 100 | 100 |

### Results

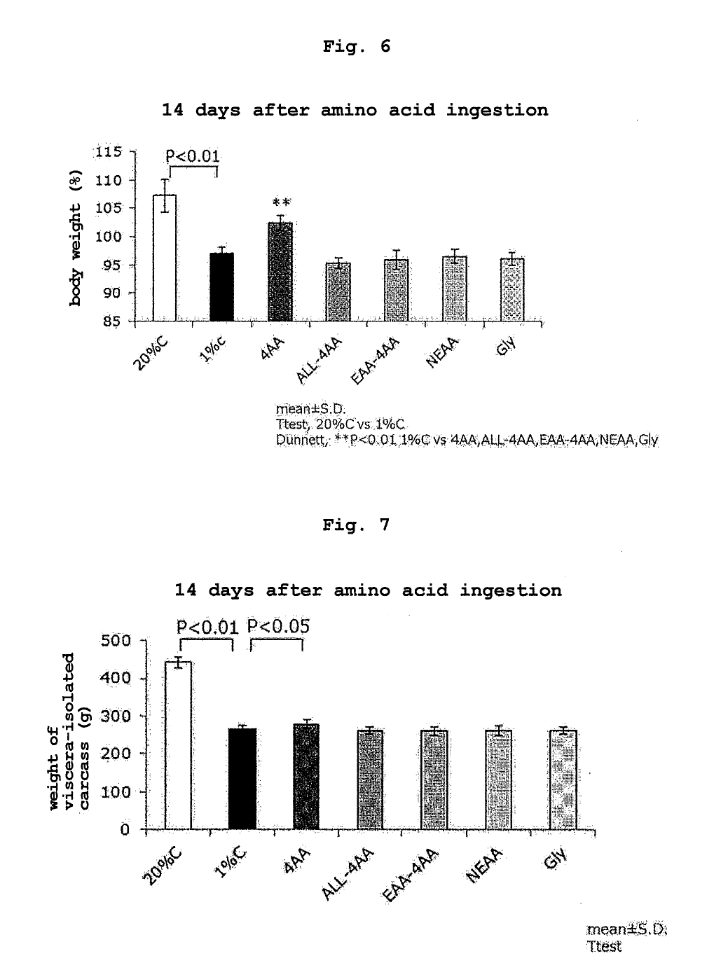

The body weight of the rats 14 days after feeding with the amino acid added diet was measured, the ratio (%) to the body weight measured on day 0 was calculated and shown in FIG. 6. In the 1% C group, the body weight decreased, whereas the body weight was significantly recovered in 4AA group. On the other hand, the body weight was not recovered in ALL-4AA group, EAA-4AA group, NEAA group and Gly group.

The weight of viscera-isolated carcass is shown in FIG. 7. The weight of viscera-isolated carcass significantly increased in 4AA group, and an increase in the muscle mass was observed.

The amino acid composition of the feeds used in Experimental Examples 2 to 5 (mass % to total free amino acid) and amino acid amount per 1 kg feed are shown in Table 21.

TABLE 21
Amino acid composition (wt % relative to total free amino acid) and amount of amino acid added per 1 kg feed

| | | | | | | | | | ALL- | EAA- | | | weight |
|---|---|---|---|---|---|---|---|---|---|---|---|---|---|
| | unit | TMIW | TMI | MIW | TIW | TI | I | T | TMIW | TMIW | NEAA | G | ratio |
| L-tryptophan | % | 11.0 | | 16.3 | 14.1 | | | | | | | | 1.0 |
| L-threonine | % | 32.3 | 36.3 | | 41.4 | 48.2 | | 100.0 | | | | | 2.9 |
| L-methionine | % | 22.1 | 24.8 | 32.5 | | | | | | | | | 2.0 |
| L-isoleucine | % | 34.6 | 38.9 | 51.2 | 44.5 | 51.8 | 100.0 | | | | | | 3.1 |
| essential, | % | | | | | | | | 100.0 | | | | |
| non-essential | | | | | | | | | | | | | |
| amino acid other | | | | | | | | | | | | | |
| than TMIW | | | | | | | | | | | | | |
| essential | % | | | | | | | | | 100.0 | | | |
| amino acid other | | | | | | | | | | | | | |
| than TMIW | | | | | | | | | | | | | |
| non-essential | % | | | | | | | | | | 100.0 | | |
| amino acid | | | | | | | | | | | | | |
| L-glycine | % | | | | | | | | | | | 100.0 | |
| total amino | g/kg | 1.15 | 1.02 | 0.78 | 0.90 | 0.77 | 0.40 | 0.37 | 1.15 | 1.15 | 1.15 | 1.15 | |
| acid amount | | | | | | | | | | | | | |

In the Table, amino acid is indicated with a single letter, T: threonine, M: methionine, I: isoleucine, W: tryptophan, G: glycine. For example, TMIW indicates a threonine, methionine, isoleucine and tryptophan added feed.

The ratio of essential and non-essential amino acids followed the general ratio of amino acid contained in casein.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An agent for the prophylaxis or improvement of frailty, comprising isoleucine and threonine as active ingredients.

2. The agent according to claim 1, further comprising at least one amino acid selected from the group consisting of tryptophan and methionine as an active ingredient.

3. The agent according to claim 1, wherein said isoleucine and threonine are present in a weight ratio 1:0.2 to 10.

4. The agent according to claim 2, wherein said tryptophan, threonine, methionine, and isoleucine are present in a weight ratio 1:0.5 to 12:0.2 to 10:0.5 to 12.

5. The agent according to claim 1, which is in a unit package form per serving comprising not less than 0.06 g of an amino acid as an active ingredient in an ingestion amount per one serving.

6. The agent according to claim 1, wherein the frailty is at least one kind of symptom selected from the group consisting of loss of muscle mass, decline in grip strength, feeling of fatigue, decrease in walking speed, decrease in the amount of physical activity, loss of motivation, depression state, delirium, dementia, sleep disorder, anxiety disorder and social withdrawal, in elderly person.

7. The agent according to claim 2, which is substantially free of an amino acid other than tryptophan, threonine, methionine and isoleucine.

8. A food or drink for the prophylaxis or improvement of frailty, which is in a unit package form per serving comprising not less than 0.04 g of isoleucine and threonine in total as an active ingredient in a weight ratio of isoleucine and threonine of 1:0.2 to 10.

9. A food or drink for the prophylaxis or improvement of frailty, which is in a unit package form per serving comprising not less than 0.06 g of 4 kinds of amino acids of tryptophan, threonine, methionine, and isoleucine in total as an active ingredient in a weight ratio of tryptophan, threonine, methionine and isoleucine of 1:0.5 to 12:0.2 to 10:0.5 to 12.

10. A kit, comprising a measuring container and a food or drink for the prophylaxis or improvement of frailty, comprising isoleucine and threonine as active ingredients, wherein said measuring container is suitable for measuring not less than 0.04 g in total of said isoleucine and threonine.

11. A kit, comprising a measuring container and a food or drink for the prophylaxis or improvement of frailty, comprising tryptophan, threonine, methionine, and isoleucine as active ingredients, wherein said measuring container is suitable for measuring not less than 0.06 g in total of the said tryptophan, threonine, methionine, and isoleucine.

12. A method for the prophylaxis and/or improvement of frailty, comprising administering an effective amount of a composition comprising isoleucine and threonine to a subject in need thereof.

13. The method according to claim 12, wherein said composition further comprises at least one amino acid selected from the group consisting of tryptophan and methionine as an active ingredient.

14. The method according to claim 12, wherein said isoleucine and threonine are present in said composition in a weight ratio 1:0.2 to 10.

15. The method according to claim 13, wherein said tryptophan, threonine, methionine, and isoleucine are present in said composition in a weight ratio 1:0.5 to 12:0.2 to 10:0.5 to 12.

16. The method according to claim 12, wherein said composition is in a unit package form per serving comprising not less than 0.06 g of an amino acid as an active ingredient in an ingestion amount per one serving.

17. The method according to claim 12, wherein the frailty is at least one kind of symptom selected from the group consisting of loss of muscle mass, decline in grip strength, feeling of fatigue, decrease in walking speed, decrease in the amount of physical activity, loss of motivation, depression state, delirium, dementia, sleep disorder, anxiety disorder and social withdrawal, in elderly person.
